(19) United States
(12) Patent Application Publication
(10) Pub. No.: US 2003/0216302 A1
(43) Pub. Date: Nov. 20, 2003

(54) Stable aqueous composition of a peptide

(75) Inventors: Subhas Balaram Bhowmick, Baroda (IN); Ritu Nitin Laddha, Baroda (IN)

Correspondence Address: ARMSTRONG,WESTERMAN & HATTORI, LLP, 1725 K STREET, NW, SUITE 1000, WASHINGTON, DC, 20006, US

(73) Assignee: SUN PHARMACEUTICAL INDUSTRIES LIMITED, Mumbai (IN)

(21) Appl. No.: 10/437,044

(22) Filed: May 14, 2003

(30) Foreign Application Priority Data
May 15, 2002 (IN) 441/MUM/2002

(51) Int. Cl. A61K 38/12; A61K 31/192

(52) U.S. Cl. 514/9; 514/568

(57) ABSTRACT

The present invention relates to a stable aqueous composition comprising desmopressin or its other pharmaceutically acceptable salts in a pharmaceutically acceptable carrier, wherein the carrier comprises a buffering agent, a parahydroxybenzoate preservative, and a cosolvent.

### FIELD OF THE INVENTION

The present invention relates to a stable aqueous composition comprising desmopressin or its other pharmaceutically acceptable salts in a pharmaceutically acceptable carrier, wherein the carrier comprises a buffering agent, a parahydroxybenzoate preservative, and a cosolvent.

### BACKGROUND OF THE INVENTION

Desmopressin, 1-(3-mercaptopropanic acid)-8-D-arginine-vasopressin is a synthetic analogue of the natural pituitary hormone 8-arginine vasopressin (ADH), an antidiuretic hormone affecting renal water conservation. It is indicated in the management of various medical conditions like irregular urination or diurea, particularly those associated with diabetes insipidus and nocturnal enuresis.

Nasal drug administration has been routinely used for administration of drugs for the upper respiratory tract, especially adrenergic agents, and is now also being used as a viable alternative for the delivery of many systemic therapeutic agents. A number of nasal dosage forms are available and include solutions, suspensions and gels. Nasal solutions are solutions prepared for nasal administration either as drops or sprays. Nasal suspensions are liquid preparations containing insoluble materials for nasal administration, primarily as drops. Nasal gels are semisolid preparations prepared for nasal application and can be for either local or systemic use, in a water soluble or water miscible vehicle. Nasal ointments are generally prepared from either water miscible/soluble or oleaginous bases. Nasal delivery system has advantages like use of lower doses, rapid local therapeutic effect, rapid systemic therapeutic blood level, rapid onset of pharmacological activity and relatively fewer side effects. The nasal administration of active substances especially peptides is a widely used method of treatment. This is because oral administration of peptide results in inactivation of the peptide in the gastrointestinal tract.

Desmopressin has been administered by the intranasal, subcutaneous, intravenous and intramuscular route in physiological saline solution. Nasal administration is an attractive route for the delivery of therapeutic peptides like desmopressin.

A problem associated with peptide drugs, especially those containing easily oxidizable sulfur bonds or sulfur bridges, is the easy degradation of their aqueous solutions. Aqueous solutions of peptides are useful as the biological activity of the peptides is often extremely high and only very small amounts of the peptide are needed for a single dose. However, these dilute aqueous solutions of the peptide are not stable at room temperature for longer periods, even when kept in sealed containers. Desmopressin is an example of such a peptide. The aqueous solution of desmopressin has to be stored at a temperature not exceeding 8° C. Storage at higher temperatures, including room temperature, results in the degradation of the peptide by hydrolytic and/or oxidative processes.

Attempts to make a composition for storage at room temperature have been made. U.S. Pat. No. 4,613,500 (Teijin Limited) discloses the use of powder nasal spray compositions that show increased stability as compared to the liquid nasal sprays. However the powder compositions also exhibit nasal mucosal irritation problems due to the presence of water-absorbing, insoluble dispersing agents that are employed to assist the absorption of the active ingredient.

U.S. Pat. No. 5,482,931 (Ferring AN) claims a stable aqueous composition for administering biologically active peptides, such as desmopressin, consisting essentially of a buffer, a quaternary amine preservative or disinfectant, and an agent for controlling osmotic pressure. The quarternary ammonium preservative used in the system of the patent prevents the degradation of the active principle at all temperatures, including room temperature, and prevents the adsorption of the active principle to the walls of the container. The preferred quaternary amine preservative according to this patent is benzalkonium chloride. However Hofmann T. et al., (HNO, Springer-Verlag, Berlin, Heidelberg, Volume 46, Issue 2, (1998), pp 146-151) have reported that benzalkonium chloride causes the irreversible suppression of the nasal ciliary motility, and should be avoided in formulations for nasal administration. The patent exemplifies formulations containing benzyl alcohol, methyl paraben, propyl paraben and chlorobutanol as the preservatives. But the shelf life of these formulations was found to be shorter than that with benzalkonium chloride.

U.S. Pat. No. 5,397,771 (Bechgaard International research & development A/S) relates to a method for administering a biologically active substance dissolved in a n-glycofurol-containing vehicle further comprising a component selected from water, vegetable oil, n-ethylene glycol and mixtures thereof Desmopressin is not exemplified in the patent. But there is nothing in the disclosure which suggests that a peptide like desmopressin would be stabilized in the formulation.

Hence, there exists a need for nasal compositions comprising desmopressin, which are stable at room temperatures, while at the same time do contain preservatives that are not irritating to the nasal mucosa and that provide an improved preservative efficacy.

### OBJECT OF THE INVENTION

It is the object of the present invention to provide a stable aqueous composition comprising desmopressin or its other pharmaceutically acceptable salts in a pharmaceutically acceptable carrier, wherein the carrier comprises a buffering agent, a parahydroxybenzoate preservative, and a cosolvent.

### SUMMARY OF THE INVENTION

It has found that preservative efficacy of desmopressin aqueous compositions containing a parahydroxybenzoate preservative is improved with the use of cosolvents. The present invention relates to a stable aqueous composition comprising desmopressin or its other pharmaceutically acceptable salts in a pharmaceutically acceptable carrier, wherein the carrier comprises a buffering agent, a parahydroxybenzoate preservative, and a cosolvent.

### DETAILED DESCRIPTION OF THE INVENTION

The present invention relates to a stable aqueous composition comprising desmopressin or its other pharmaceutically acceptable salts in a pharmaceutically acceptable carrier, wherein the carrier comprises a buffering agent, a parahydroxybenzoate preservative, and a cosolvent.

No particular limitation is imposed on the peptide for use in the composition according to the present invention insofar as it is a peptide having pharmacological effects. Its specific examples can include calcitonin, insulin, proinsulins, epidermal growth factors, growth hormones, somatomedin C, somatostatin, granulocyte macrophage colony-stimulating factor, colony-stimulating factors, erythropoietin, interferons, interleukins, atrial natriuretic peptides, parathyroid hormones, superoxide dismutases, tissue plasminogen activators, antithrombins, blood coagulation-factor, blood coagulation-factor, protein C, hirudine, hepatitis vaccine, endorphins, ACTH-releasing hormone, neurotensin, angiotensin, transferrin, endothelin, vasopressin, desmopressin, terlipressin, atosiban, carbetocin, luteinizing hormone, luteinizing hormone-releasing hormone, triptorelin, prolactin, glucagon, gastrin, secretin, urokinase, vasoactive intestinal polypeptide and the like. It is preferred for the peptide or peptide analog to be oxytocin or vasopressin, or their analogs and derivatives, such as desmopressin (1-(3-mercaptopropanic acid)-8-D-arginine-vasopressin), terlipressin (N-.alpha.-triglycyl-8-lysine)-vasopressin), atosiban ((Mpa¹, D-Tyr(Et)², Thr⁴, Orn⁸)-oxytocin), carbetocin ((1-desamino-1-monocarba-2(0-methyl)-tyrosine)-oxytocin), and the like. The most preferred peptide is desmopressin or its other pharmaceutically acceptable salts which are used in the management of various medical conditions like irregular urination or diurea, particularly those associated with diabetes insipidus and nocturnal enuresis.

The buffering agent used in the stable nasal composition of the present invention may be any pharmaceutically acceptable pH-adjusting agent, known to a person skilled in the art. The buffering agent may be selected from a group consisting of organic acids and its salts, mineral acids, alkali metal phosphates, carbonates, borates, hydroxides, base and the like and mixtures thereof. Preferably selected from a group consisting of lactic acid, citric acid, tartaric acid, phosphoric acid, acetic acid, hydrochloric acid, nitric acid, sodium or potassium metaphosphate, sodium or potassium phosphate, sodium or potassium acetate, ammonia, sodium carbonate, sodium or potassium hydroxide, dibasic sodium phosphate, sodium borate, and the like and mixtures thereof. A combination of disodium hydrogen ortho phosphate dihydrate and tartaric acid is used as the preferred buffering agent. This buffer combination also functions as the osmotic agent in the composition. It is used in an amount ranging from about 0.01% to about 0.5% w/v. The composition may optionally contain osmotic pressure regulating agents.

The preservative(s) incorporated in the present composition is/are selected from the group comprising parahydroxybenzoates such as methyl p-hydroxybenzoate (methyl paraben), ethyl p-hydroxybenzoate (ethyl paraben), propyl p-hydroxybenzoate (propyl paraben), butyl p-hydroxybenzoate (butyl paraben), isobutyl p-hydroxybenzoate (isobutyl paraben), isopropyl p-hydroxybenzoate (isopropyl paraben), benzyl p-hydroxybenzoate (benzyl paraben) and the like and mixtures thereof. The preferred preservative used in the process of the present invention is a paraben or a mixture of parabens, more a combination of methyl p-hydroxybenzoate and propyl p-hydroxybenzoate, the mixture being used in an amount ranging from about 0.001% w/v to about 0.5% w/v.

The cosolvent that may be used in the present invention is pharmaceutically acceptable, more particularly ophthalmically acceptable. Cosolvents used in the present invention comprise alcohols, polyvinyl alcohols, propylene glycol, polyethylene glycols and derivatives thereof, glycerol, sorbitol, polysorbates, ethanol and the like and mixtures thereof. More preferably, the cosolvent may be a glycol selected from the group consisting of ethylene glycol, poly(ethylene glycol), propylene glycol, ethylene glycol derivatives, poly(ethylene glycol) derivatives, propylene glycol derivatives and the like and mixtures thereof. The polyethylene glycols are available in different grades based on their molecular weight and are commonly referred to by their abbreviated synonym PEG followed by a number which indicates the average molecular weight of the polymer. The polyethylene glycol grades 200-600 are liquids whilst grades 1000 and above are solid at ambient temperatures. The preferred cosolvent used is in the present invention is propylene glycol. Propylene glycol helps in improving the efficacy of the parahydroxybenzoate preservatives used. It is used in an amount ranging from about 1.0% w/v to about 5.0% w/v. Propylene glycol also contribute substantially as a penetration enhancer for the drug.

Chelating agents used in the nasal composition of the present invention may be selected from a group comprising edetic acid, edetic acid salts like disodium edetate, sodium edetate, edetate calcium disodium, and trisodium edetate, malic acid and the like, and mixtures thereof.

The composition of the present invention may be prepared by a simple process comprising mixing the desmopressin acetate in an aqueous solution of the buffer and the preservatives. The tartaric acid, disodium hydrogen ortho phosphate dihydrate and disodium edetate were dissolved in Water for Injection. The methyl paraben and propyl paraben were dissolved in propylene glycol. The preservative solution was added to the buffer solution. The desmopressin acetate was then added to the above solution and stirred. The volume was made up by Water for Injection. The solution was filtered under nitrogen pressure through a 0.45 μm Nylon 66 membrane filter and filled in amber USP Type I vials.

The invention is further illustrated by the following examples, which are by no means intended to limit the scope of the invention but are given by way of illustration.

### EXAMPLES 1-3

Three preferred embodiments of the present invention are given in Table 1 below.

TABLE 1

| | | Amount (mg) | | |
|---|---|---|---|---|
| | | (Percent (w/v)) | | |
| S. No. | Ingredients | Example 1 | Example 2 | Example 3 |
| 1 | Desmopressin Acetate | 0.1 mg | 0.1 mg | 0.1 mg |
| | | (0.01%) | (0.01%) | (0.01%) |
| 2 | Methyl Paraben | 1.8 mg | 1.8 mg | 1.8 mg |
| | | (0.18%) | (0.18%) | (0.18%) |
| 3 | Propyl Paraben | 0.2 mg | 0.2 mg | 0.2 mg |
| | | (0.02%) | (0.02%) | (0.02%) |
| 4 | Propylene Glycol | 15 mg | 16 mg | 20 mg |
| | | (1.50%) | (1.60%) | (2.0%) |
| 5 | Disodium Hydrogen ortho | 3.4 mg | 3.0 mg | 3.4 mg |
| | Phosphate Dihydrate | (0.34%) | (0.30%) | (0.34%) |
| 6 | Tartaric acid | 1.5 mg | 1.3 mg | 1.5 mg |
| | | (0.15%) | (0.13%) | (0.15%) |
| 7 | Disodium Edetate | 0.2 mg | 0.2 mg | 0.2 mg |
| | | (0.02%) | (0.02%) | (0.02%) |
| 8 | Water for Injection, | 1 ml | 1 ml | 1 ml |
| | quantity sufficient to make | | | |

The tartaric acid, disodium hydrogen ortho phosphate dihydrate and disodium edetate were dissolved in Water for Injection. The methyl paraben and propyl paraben were dissolved in propylene glycol. The preservative solution was added to the buffer solution and commercial nitrogen gas filtered through 0.45 μm nylon membrane was purged through it for 10-15 minutes. The desmopressin acetate was then added to the above solution and stirred using paddle stirrer. The volume was made up by Water for Injection. The solution was filtered under nitrogen pressure using silicone tubing through a 0.45 μm Nylon 66 membrane filter. The solution was filled in amber USP Type I vials with pre- and post-nitrogen purging. The metered dose pump was snapped onto the filled vial with the snap-on equipment followed by gentle fixing of the actuators and protective caps and suitably packaged.

The vials were filled with formulation described in Example 1 and stored at 25° C., 40° C. and in a refrigerator, for a period of six months. The vials were placed in the inverted and upright position for each condition. The desmopressin acetate was analyzed by stability indicating spectrophotometric analysis, at 420 nm. The results of the accelerated stability study are given in Table 2 below. Results indicated that the composition was stable at room temperature.

TABLE 2
Results of stability studies on the composition of Example 1

| | Assay | Degradation | |
|---|---|---|---|
| Condition | (% of label claim) | Individual (%) | Total (%) |
| Limit | 90.0-110.0 | Not more | Not more |
| | | than 1% | than 3% |
| Initial | 97.61 | | 0.62 |
| 25° C. 1M-U | 101.48 | 0.24 | 0.24 |
| 25° C. 1M-I | 102.85 | 0.24 | 0.27 |
| 25° C. 2M | 101.50 | 0.27 | 0.27 |
| 25° C. 3M-U | 98.04 | 0.30 | 0.30 |
| 25° C. 1M-I | 97.95 | 0.26 | 0.30 |
| 25° C. 6M | 97.22 | 0.44 | 0.55 |
| 40° C. 1M-U | 100.11 | 0.79 | 0.84 |
| 40° C. 1M-I | 100.34 | 0.78 | 0.83 |
| 40° C. 2M | 101.40 | 0.98 | 1.19 |
| 40° C. 3M-U | 94.14 | 1.50 | 1.90 |
| 40° C. 3M-I | 94.65 | 1.40 | 1.80 |
| 40° C. 6M | 91.83 | 2.30 | 2.90 |
| Fridge | 98.02 | 0.64 | 0.88 |

1M: 1 Month, 2M: 2 Months, 3M: 3 Months, 6M: 6 Months, U: Upright position of the vial, I: Inverted position of the vial

The reduction in urine output of rats was tested in comparison to DDAVP® Nasal Spray (nasal spray marketed by AVENTIS, containing 0.01% desmopressin acetate) as the reference. Formulations of example 1 was used as the test. Wistar rats of either sex weighing 200-300 gm were used in the study. The animals were maintained on 12 hour light-dark cycle and had free access to food and water. In the control group, rats were given intranasal administration of vehicle (6 μl/100 gm) and kept fasted in metabolic cages. They had free access to water for 24 hours. In the treated groups, the animals were given intranasal administration of desmopressin nasal spray formulation (reference and teast) at a dose of 6 μg/Kg of desmopressin. These animals were kept in metabolic cages as in the control group. After 24 hours, cumulative urine volume was measured and compared with control. The results of the study are given in Table 3 below.

TABLE 3
Comparison of reduction in urine output in rats

| | Urine output |
|---|---|
| Group | (ml/100 gm) |
| Control (n = 11) | 5.3 ± 0.23 |
| DDAVP ®, Batch No YD4641, April 1999 (0.01% | 2.6 ± 0.46 |
| desmopressin acetate) (n = 6) | |
| DDAVP ®, Batch No AI5880, January 2000 | 2.25 ± 0.20 |
| (0.01% desmopressin acetate) (n = 6) | |
| Example 1 (0.01% desmopressin acetate) (n = 6) | 2.2 ± 0.08 |
| Example 2 (0.01% desmopressin acetate) (n = 6) | 2.1 ± 0.12 |
| Example 3 (0.01% desmopressin acetate) (n = 6) | 2.5 ± 0.19 |
| n = number of rats used | |

The results of the study indicate that the three compositions obtained by the process of the present invention produced a marked reduction (more than 50%, as compared to vehicle treated control) in the cumulative urine output for 24 hours in rats, and have an efficacy equivalent to the standard DDAVP® formulation.

1. A stable aqueous composition comprising desmopressin or its other pharmaceutically acceptable salts in a pharmaceutically acceptable carrier, wherein the carrier comprises a buffering agent, a parahydroxybenzoate preservative, and a cosolvent.

2. A stable aqueous composition as claimed in claim 1, wherein the buffering agent is selected from a group consisting of organic acids and its salts, mineral acids, alkali metal phosphates, carbonates, borates, hydroxides, base and the like and mixtures thereof.

3. A stable aqueous composition as claimed in claim 2, wherein the buffering agent is selected from a group consisting of lactic acid, citric acid, tartaric acid, phosphoric acid, acetic acid, hydrochloric acid, nitric acid, sodium or potassium metaphosphate, sodium or potassium phosphate, sodium or potassium acetate, ammonia, sodium carbonate, sodium or potassium hydroxide, dibasic sodium phosphate, sodium borate, and the like and mixtures thereof.

4. A stable aqueous composition as claimed in claim 3, wherein the buffering agent used is a combination of disodium hydrogen ortho phosphate dihydrate and tartaric acid.

5. A stable aqueous composition as claimed in claim 4, wherein the buffering agent is used in an amount ranging from about 0.01% to about 0.5% w/v.

6. A stable aqueous composition as claimed in claim 1, wherein the parahydroxybenzoate preservative is selected from methyl p-hydroxybenzoate (methyl paraben), ethyl p-hydroxybenzoate (ethyl paraben), propyl p-hydroxybenzoate (propyl paraben), butyl p-hydroxybenzoate (butyl paraben), isobutyl p-hydroxybenzoate (isobutyl paraben), isopropyl p-hydroxybenzoate (isopropyl paraben), benzyl p-hydroxybenzoate (benzyl paraben) and the like and mixtures thereof.

7. A stable aqueous composition as claimed in claim 6, wherein the preservative used is a mixture of methyl p-hydroxybenzoate and propyl p-hydroxybenzoate.

8. A stable aqueous composition as claimed in claim 7, wherein the mixture of methyl p-hydroxybenzoate and propyl p-hydroxybenzoate is in an amount ranging from about 0.001% w/v to about 0.5% w/v.

9. A stable aqueous composition as claimed in claim 8, wherein the methyl p-hydroxybenzoate is used in amounts of about 0.18% w/v and propyl p-hydroxybenzoate is used in amounts of about 0.02% w/v.

10. A stable aqueous composition as claimed in claim 1, wherein the cosolvent comprises alcohols, polyvinyl alcohols, propylene glycol, polyethylene glycols and derivatives thereof, glycerol, sorbitol, polysorbates, ethanol and the like and mixtures thereof.

11. A stable aqueous composition as claimed in claim 10, wherein the cosolvent used is propylene glycol.

12. A stable aqueous composition as claimed in claim 11, wherein the propylene glycol is used in an amount ranging from about 1.0% w/v to about 5.0% w/v.

13. A stable aqueous composition as claimed in claim 1, wherein the composition further comprises a chelating agent.

14. A stable aqueous composition as claimed in claim 13, wherein the chelating agent is selected from a group comprising edetic acid, edetic acid salts like disodium edetate, sodium edetate, edetate calcium disodium, and trisodium edetate, malic acid and the like, and mixtures thereof.
